(19) United States
(12) Patent Application Publication
(10) Pub. No.: US 2007/0178463 A1
(43) Pub. Date: Aug. 2, 2007

(54) Micro-array substrate for biopolymer, hybridization device, and hybridization method

(76) Inventors: Takeo Tanaami, Tokyo (JP); Hideo Tashiro, Wako-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/590,455

(22) PCT Filed: Feb. 17, 2005
(86) PCT No.: PCT/JP05/02440

§ 371 Date: Aug. 24, 2006

(30) Foreign Application Priority Data
Mar. 1, 2004 (JP) 2004-056237

(51) Int. Cl. C12Q 1/68; C12M 3/00

(52) U.S. Cl. 435/6; 435/287.2

(57) ABSTRACT

The invention provides a substrate for biopolymer hybridization, a biopolymer hybridization device, and a biopolymer hybridization method capable of: speeding up hybridization of a biopolymer, by means of dielectrophoresis or electrophoresis by applying an alternating voltage or a direct voltage to a planar electrode, so as to generate an electric field; and reading the hybridized biopolymer by means of a laser or the like.

### BACKGROUND OF THE INVENTION

1. Field of the Invention

The present invention relates to micro-array substrate for biopolymers such as DNA and RNA, a hybridization device using this substrate, and a hybridization method for speeding up hybridization using this device.

2. Description of Related Art

Conventionally, in gene diagnosis, specification of pathogens, detection of single nucleotide polymorphism, and the like, in order to. detect a nucleic acid (target nucleic acid) serving as a test object, there is widely used a hybridization between a probe nucleic acid and the target nucleic acid. Recently, a DNA chip and a DNA micro-array having a large number of probe nucleic acids immobilized on a substrate are practically in use, and hybridization is used for detecting the target nucleic acid.

In production of a biopolymer micro-array (for example, a DNA chip or a DNA micro-array), it is needed to align and immobilize a large number of probe DNAs respectively as spots on a substrate. An example of a method of immobilizing DNA includes a method of uniting thiol with a single strand DNA, and immobilizing the thiolated single strand DNA for example on a metal substrate.

The target DNA serving as the test object is made to act on the immobilized probe DNA, to detect the presence/absence of hybridization therebetween. The presence/absence of hybridization can be detected for example by using the fluoroscopic method, involving measuring fluorescence from a spot of fluorescent labeled target DNA hybridized with the probe DNA.

A spotting-type DNA micro-array is produced by putting droplets containing probe DNA on a substrate and drying it (refer to Non Patent Document 1). Therefore, an advantage is that it can be produced at low cost while a disadvantage is that evenness of DNA immobilized on the substrate can not be guaranteed. That is, the disadvantage is that the size and the shape of the DNA detection spot vary.

Furthermore, in the case of the spotting-type DNA micro-array, due to the presence of a solid-phasing agent adhered around the DNA detection spot, the target DNA is nonspecifically absorbed on the substrate, increasing the noise, and causing a problem of decreasing S/N ratio (refer to Non Patent Document 1).

At the time of measuring fluorescence, an operation called gridding which specifies the fluorescent part is performed. Gridding means an operation for inputting the number of spots and the gap between spots lengthwise and widthwise on the array and the diameter of the spots, and enclosing the spots by a circle (refer to Non Patent Document 1). However, if the stamp shape and the position of the spots are not stable, the gridding operation at the time of measuring fluorescence takes a long time, and an accurate analysis becomes difficult.

Moreover, in the gridding, if the position of the spots is displaced, the spots can not be accurately enclosed. Therefore, the software that performs gridding is installed with a function to automatically correct the position. However, not all operations are automatic, a starting point of the spots has to be manually set, and the grid of all spots has to be confirmed and corrected by the eye. This operation is very complicated and takes a very long time if the number of DNA spots is more than several thousands, which becomes a factor for slowing down the analysis speed.

On the other hand, hybridization of the probe DNA immobilized on the substrate and the target DNA serving as a sample normally takes over ten hours. Furthermore, a large amount of sample is required for hybridization. As a result, such a long hybridization time and preparation of a large amount of samples requires enormous time, cost, and labor. In particular, if a low expressed gene is analyzed, an extremely large amount of sample is required.

[Non Patent Document 1]“DNA micro-array Practice Manual which surely gives data, Fundamental Principles, From Chip Production Technique to Bioinformatics” First edition, Yodosha. Co., Ltd. 1 Dec., 2002, p. 19-21, 35, 106-108.

### DISCLOSURE OF INVENTION

An object of the present invention is to address such problems. The present invention provides a substrate for biopolymer hybridization, a biopolymer hybridization device, and a hybridization method capable of: speeding up hybridization of a biopolymer, by means of dielectrophoresis and electrophoresis by applying an alternating voltage or a direct voltage to a planar electrode, so as to generate an electric field; and reading the hybridized biopolymer by means of a laser or the like.

In order to achieve such an assignment, the present invention provides the following.

(1) In a micro-array substrate for biopolymer detection

a micro-array substrate for a biopolymer, wherein a pair of two conduction paths connected to a direct-current or alternating-current source are installed on the substrate, in a part of a conduction path pattern is arranged the two conduction paths in proximity to each other to a degree such that an electric field distribution between the conduction paths becomes locally stronger, and probe molecules for biopolymer detection are immobilized on the conduction paths or close to its proximity part.

(2) In a micro-array substrate for biopolymer detection

a micro-array substrate for a biopolymer, wherein a pair of two conduction paths connected to a direct-current or alternating-current source are installed on the substrate, in a part of a conduction path pattern is arranged the two conduction paths in proximity to each other to a degree such that an electric field distribution between the conduction paths becomes locally stronger, and probe molecules for biopolymer detection are immobilized on the conduction paths of the proximity part in an opposed substrate arranged opposite to the substrate, or close to its proximity part.

According to such a structure, by applying an alternating voltage or a direct voltage between the conduction paths, to generate an electric field of a distribution which becomes locally stronger between the conduction paths arranged in proximity to each other, then the biopolymer can be subjected to dielectrophoresis or electrophoresis in the part arranged with the conduction paths, and readily concentrated.

(3) A micro-array substrate for a biopolymer having two or more proximity parts of conduction paths.

(4) A micro-array substrate for a biopolymer according to (1) or (2), wherein a glass, a plastic, or a ceramic is used for the substrate, and the conduction paths are formed on the glass substrate by means of etching or printing.

(5) A micro-array substrate for a biopolymer according to any one of (1) to (4), wherein the conduction paths are insulated from a solution in areas other than an area immobilized with the probe molecules.

(6) A micro-array substrate for a biopolymer according to any one of (1) to (5), having an electrode for detecting the presence/absence of hybridization after hybridization, separately from the conduction paths.

(7) A biopolymer hybridization device comprising a micro-array substrate for biopolymers according to any one of (1) to (6), and a power source for applying either an AC voltage or DC voltage to two conduction paths set on the substrate, wherein

a voltage is applied from this power source to the conduction paths to generate an electric field, so that a sample biopolymer target contained in a solution on the substrate can be subjected to dielectrophoresis or electrophoresis, along this electric field.

According to such a structure, by applying an alternating voltage or a direct voltage between the conduction paths from the power source, to generate an electric field of a distribution which becomes locally stronger between the conduction paths arranged in proximity to each other, then the biopolymer can be subjected to dielectrophoresis or electrophoresis in the part arranged with the conduction paths, and readily concentrated, and a hybridization device capable of speeding up hybridization can be readily realized.

(8) A biopolymer hybridization device according to (7), wherein a cover substrate formed from a transparent material is provided opposite to a substrate surface set with the conduction paths, so that fluorescence from a hybridized biopolymer with fluorescent labeling can be observed through this cover substrate.

(9) A biopolymer hybridization device according to (7), wherein the conduction paths are formed on a cover substrate formed from a transparent material, so that fluorescence from the hybridized biopolymer with fluorescent labeling can be observed from the back face of this cover substrate.

(10) A method of performing hybridization of a biopolymer by using the device according to any one of (7) to (9), comprising applying an alternating voltage or a direct voltage output from the power source between the conduction paths, to generate an electric field, so that a sample biopolymer target that is spontaneously dispersed in a solution is concentrated in the vicinity of the conduction paths by dielectrophoresis or electrophoresis.

According to this method, hybridization can be readily accelerated.

(11) A hybridization method for a biopolymer according to (10), comprising detecting the sample biopolymer target by means of fluorescent signals or electrical current value, after hybridization.

As described above, the present invention has the following effects.

(1) There may be readily realized respectively: a micro-array substrate for a biopolymer wherein an alternating voltage or a direct voltage is applied between two-pole conduction paths (hereunder, these conduction path parts are called planar electrodes) provided on a substrate in proximity to each other, to generate an electric field in the vicinity of the planar electrodes, and thereby the biopolymer can be concentrated in the vicinity of the planar electrodes; a hybridization device using this micro-array substrate; and a method of speeding up hybridization.

(2) Moreover, in such a hybridization device, a cover substrate arranged opposite to the substrate surface provided with the planar electrode surface, is formed from a transparent material, and fluorescence from the hybridized biopolymer with fluorescent labeling can be readily read by a reader by a conventional laser or the like, through the cover substrate, providing an advantage in that a conventional reader can be utilized as it is.

(3) The substrate of the present invention is produced merely by attaching a planar electrode to a conventional substrate, and a biopolymer micro-array (such as a DNA chip) can be readily produced with a low production cost.

(4) This planar electrode pattern is metal with a high reflectance, and thus gridding becomes possible readily by measuring the reflected image.

### BRIEF DESCRIPTION OF THE DRAWINGS

FIG. 1 is a block diagram of an embodiment showing a part of a biopolymer hybridization device according to the present invention.

FIG. 2 shows the shape of a planar electrode.

FIG. 3 shows an example of biopolymer spots.

FIG. 4 shows another example of biopolymer spots.

FIG. 5 shows another example of setting of the planar electrode and probe DNA.

FIG. 6 shows still another example of setting of the planar electrode and probe DNA.

FIG. 7 shows yet another example of setting of the planar electrode and probe DNA.

FIG. 8 shows yet another example of setting of the planar electrode and probe DNA.

### BRIEF DESCRIPTION OF THE REFERENCE SYMBOLS

1. Biopolymer hybridization device
2. Substrate
3, 3₁, 3₂, 3₃. Planar electrode
4, 4₁, 4₂, 4₃. Planar electrode
3a, 4a. Lead wire
5. Cover substrate
6. Probe DNA
6₁, 6₂, 6₃, 6₄. Biopolymer spots
7. Sample target DNA
8. Solution
10. Power source
20. Object lens of reader

### DETAILED DESCRIPTION OF THE PREFERRED EMBODIMENTS

Hereunder is a detailed description of the present invention with reference to the drawings.

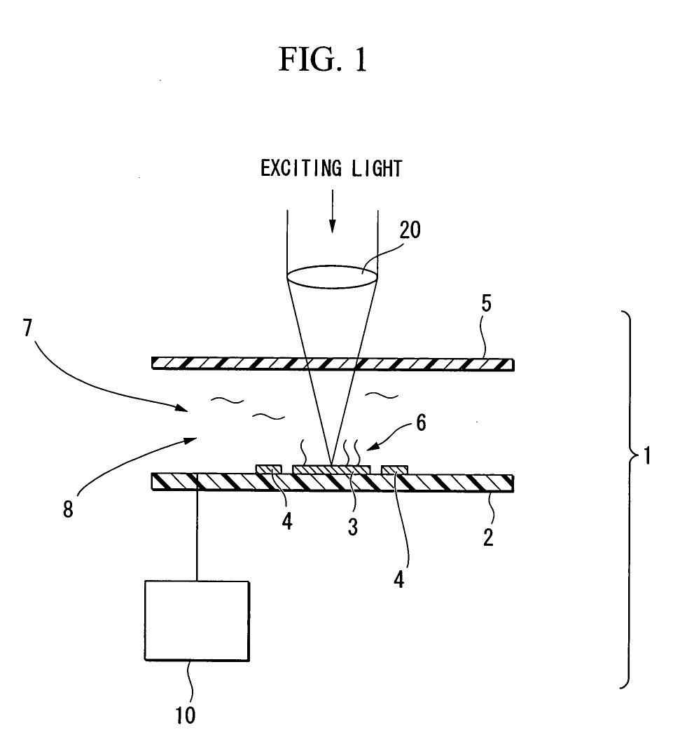

FIG. 1 is a block diagram of an embodiment showing a part of the biopolymer hybridization device according to the present invention, which also shows an object lens provided in a reader for reading the fluorescent labeling of a hybridized biopolymer.

This embodiment is described using DNA as an example of the biopolymer.

In FIG. 1, reference symbol 1 denotes a biopolymer hybridization device, and reference symbol 20 denotes an object lens of a reader. The biopolymer hybridization device 1 comprises: a substrate 2; planar electrodes 3 and 4 serving as a two-pole conduction path part attached on the top face of the substrate 2, where the conduction paths are arranged especially in proximity; a cover substrate 5 formed from a transparent material; and a power source 10 for applying an electric field between the planar electrodes 3 and 4.

On the top face of the planar electrodes 3 and 4 is immobilized a probe DNA 6. A space between the substrate 2 and the cover substrate 5 is filled with a solution 8 containing a sample target DNA 7. The structure is such that the substrate 2 and the cover substrate 5 are enclosed by side walls (not shown) to form a sealed container, so as to prevent the outflow of the solution 8. Moreover, the drawing shows the planar electrodes 3 and 4 and a probe DNA 6 related to one site, however such planar electrodes are arranged in a plurality of sites in predetermined intervals on one DNA chip or DNA micro-array.

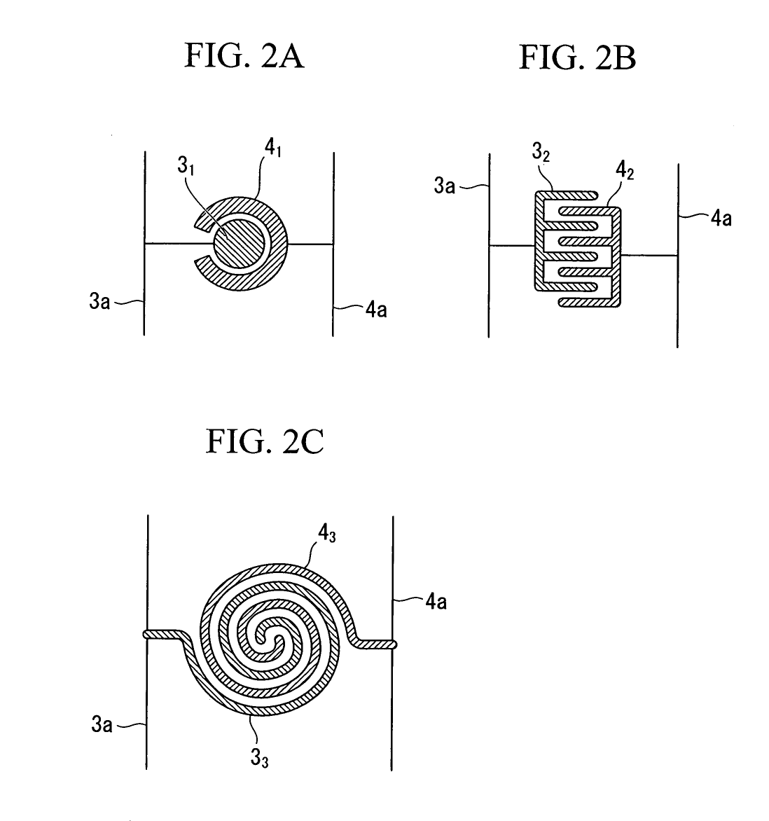

The planar electrodes 3 and 4 are arranged in proximity position so that the two poles are not in contact with each other on the substrate 2. The shapes as shown in FIG. 2A to FIG. 2C of FIG. 2 can be used.

FIG. 2A is a planar electrode comprising a circular electrode and an annular electrode surrounding this, wherein the circular electrode 3₁, and the annular electrode 4₁ are connected to the power source 10 respectively through lead wires 3a and 4a. Moreover, FIG. 2B is a pectinate planar electrode that is mutually arranged in a nest, wherein the electrodes 3₂ and 4₂ are connected to the power source 10 respectively through the lead wires 3a and 4a. Furthermore, FIG. 2C is an electrode formed mutually in a spiral, wherein the electrodes 3₃ and 4₃ are connected to the power source 10 respectively through the lead wires 3a and 4a.

The planar electrode in such a shape is attached to the surface of the substrate 2. However, it may be produced in the following manner. A slide glass having a polished surface is used as the substrate 2. The glass surface is deposited with gold by means of vacuum deposition. A resist is coated thereon and baked. Then, the slide glass is irradiated with ultraviolet radiation through a photomask by a UV exposure device. After the irradiation, development is performed and a resist pattern in the electrode shape as shown in FIG. 2 is formed on the gold surface.

The gold surface other than the resist pattern is etched by a gold etchant. By so doing, a glass substrate having a gold pattern in the electrode shape according to the photomask can be produced. The lead wire can be produced by patterning in the same manner.

The operation in such a structure is described below. The probe DNA 6 is previously stamped and immobilized on the electrode surface. For example, in FIG. 2A, probe DNA is spotted in the circular part of the electrode 3₁, and immobilized on this circular electrode. The space between the substrate 2 and the cover substrate 5 is filled with the solution 8 containing the fluorescent labeled sample target DNA 7. Then, AC voltage is applied between the planar electrodes 3₁ and 4₁ by the power source 10. As a result, the electric field density between the electrodes 3₁ and 4₁ is increased, and the negatively charged sample target DNA 7 that is spontaneously dispersed in the solution 8 is attracted to the vicinity of the electrode parts 3₁ and 4₁ by dielectrophoresis, to be concentrated.

As a result, the hybridization between the probe DNA 6 immobilized to the electrode part and the sample target DNA 7 can be promoted. Such a promoting effect by means of AC voltage is apparent from for example, from “Development of next generation DNA micro-array system —enhancement effect of hybrid formation in MESA type array” (speaker: Kohei Kasai, Tetsu Hatakeyama, Takayuki Shimamura, Yasumitsu Kondo, Tomoko Tashiro, and Hideo Tashiro) presented at the 26th Annual Meeting of the Molecular Biology Society of Japan held on 10th-13th Dec., 2003 at Kobe International Exhibition Hall.

After the hybridization, unhybridized sample target DNA is washed out together with the solution 8, and the sample target DNA 7 hybridized with the probe DNA is irradiated with exciting light (such as laser light) through the cover substrate 5 of a transparent window from the reader side. The fluorescence emitting from the fluorescent labeling enters through the cover substrate 5 into the object lens 20, and is read by the reader. In this manner, the sample target DNA 7 hybridized with the probe DNA can be measured.

For reading of the fluorescent labeling DNA, there may be used a confocal scanning microscope, a scanless multi-beam type reader, and the like.

The voltage to be applied to the electrodes 3 and 4 may be either AC or DC. If an AC voltage is applied as in the above embodiment, bubbles or the like are often generated due to electrolysis in the solution containing the sample target DNA 7 with a low frequency. Therefore a high frequency AC is preferably used.

On the other hand, if a DC voltage is applied, when a high voltage is applied, the solution containing the sample target DNA 7 is electrolyzed due to the high voltage, causing concern of bubbles and the like. Therefore a low voltage is preferably used. The negatively charged sample target DNA 7 is collected to the electrode on the positive side.

Regarding the power source 10, an alternating-current or direct-current source is used according to whether the voltage to be applied is AC or DC. Alternatively, there may be used a power source which can selectively output either one of AC voltage or DC voltage by setting.

The present invention is not limited to the above embodiment, and many other alterations or modifications can be made without departing from the sprit of the present invention.

For example, a plurality of the proximity electrode portions of FIG. 2A to FIG. 2C may be set in an array form on the substrate. As a result, a large number of DNA can be analyzed at the same time.

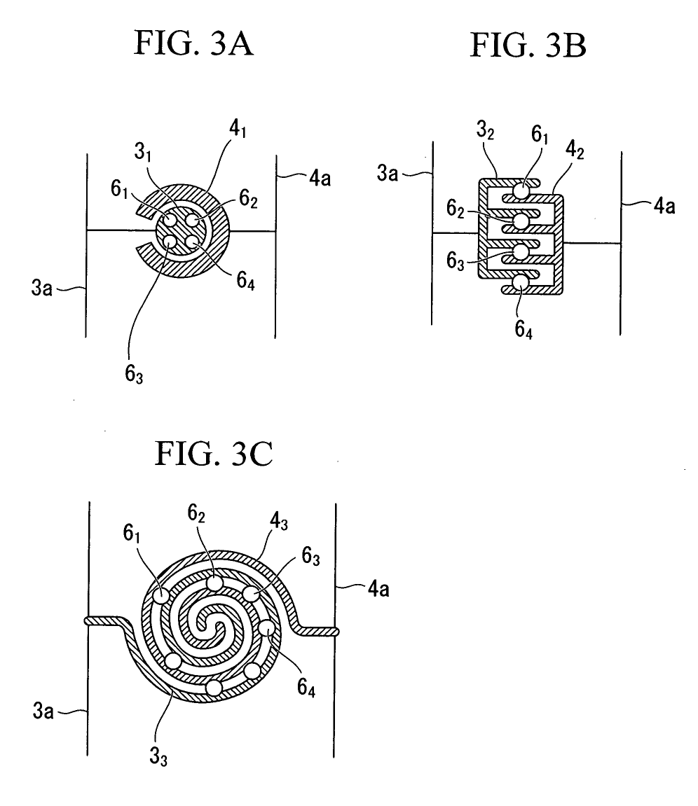

Moreover, as shown in FIG. 3A to FIG. 3C, a plurality of types of biopolymer spots can be respectively and separately spotted on one proximity electrode part.

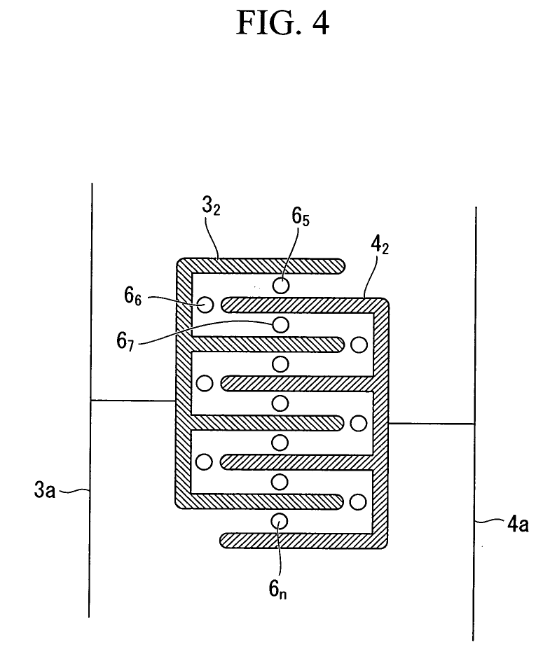

Furthermore, as shown in FIG. 4, the probe DNA 6₅, 6₆, 6₇. . . 6ₙ may be spotted not on the planar electrode but in the vicinity of the planar electrode. In this case, since the target DNA 7 is present in high concentration in the vicinity of the electrode, the hybridization is accelerated.

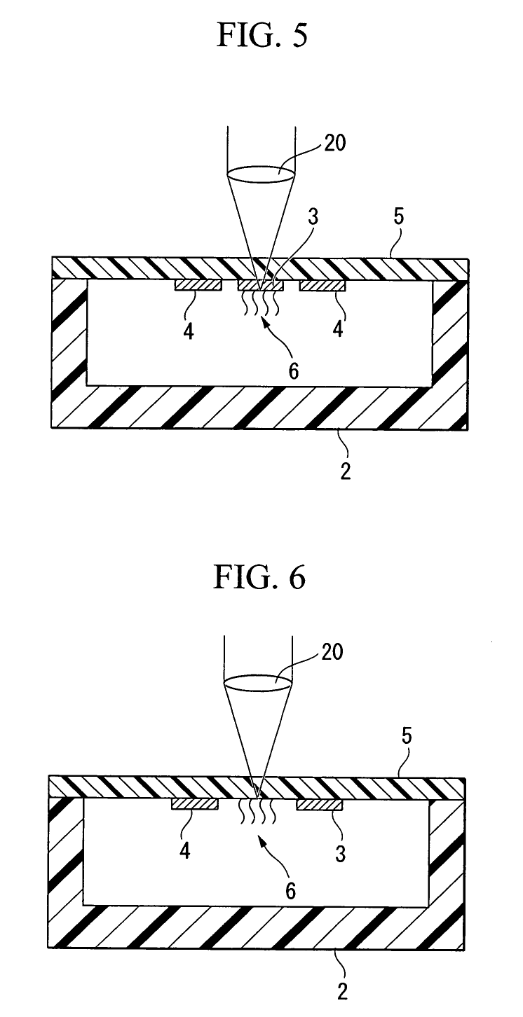

Moreover, as shown in FIG. 5, the planar electrodes 3 and 4 and the probe DNA 6 may be set on the side of the transparent cover substrate 5 for reading fluorescence. In this case, the probe DNA 6 is immobilized on the transparent planar electrode.

Furthermore, as shown in FIG. 6, the probe DNA 6 may be set in the vicinity of the planar electrode. In this case, the planar electrode need not be transparent.

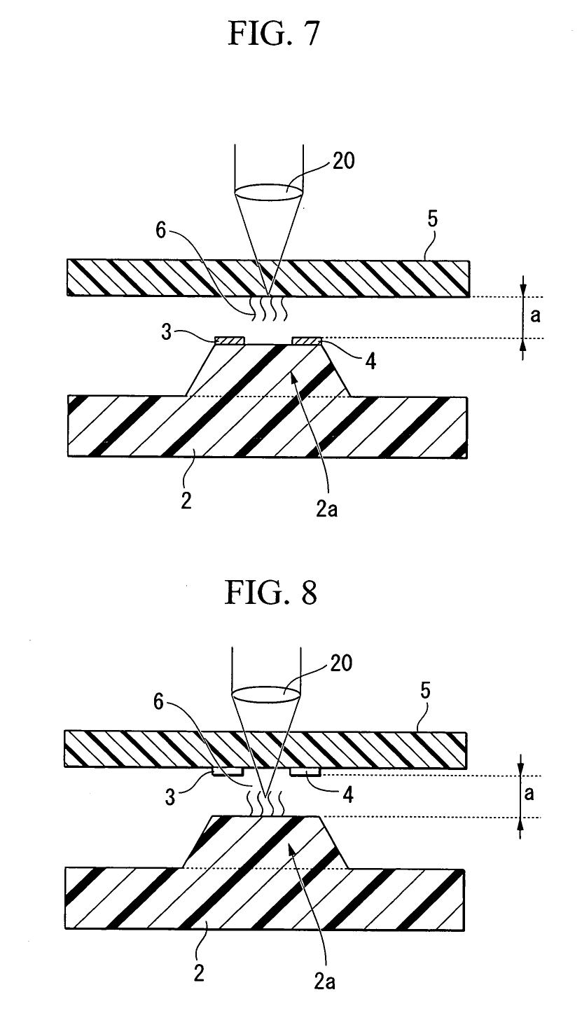

Moreover, the arrangement may be as shown in FIG. 7 and FIG. 8. In either drawing, as the substrate 2, there is used a substrate 2 having a structure where a projection 2a in a cylindrical shape with a flat top or a quadratic prism shape is on a plate. The planar electrode and the probe DNA are arranged on the opposed faces. That is, in FIG. 7, the top of the projection 2a is attached with the planar electrodes 3 and 4, and the probe DNA 6 is immobilized to the bottom of the transparent cover substrate 5. In FIG. 8, the planar electrodes 3 and 4 are attached to the bottom of the transparent cover substrate 5, and the probe DNA 6 is attached to the top of the projection 2a.

In this case, a gap a, that is the gap a between the cover substrate 5 and the planar electrodes 3 and 4 in FIG. 7, and the gap a between the planar electrodes 3 and 4 and the projection 2a in FIG. 8, is preferably narrower.

Moreover, electrolysis may occur from the lead wire other than the proximity part, however it may be insulated from the solution by a structure where areas other than the area immobilized with the biopolymer (other than the immobilized site) is covered with a nonconductive film.

Moreover, the detection may be performed in addition to by using the previously fluorescent labeled target DNA or the like, by a method of inserting an intercalator type reagent between a double strand after hybridization so as to detect by means of fluorescent signals or electrical current value. Furthermore, the detection may be performed not by fluorescence but by absorbance or phosphorescence.

In the case of current detection, in addition to the conduction paths for hybridization, there may be separately set an exclusive electrode for detection and a detection circuit.

### INDUSTRIAL APPLICABILITY

According to the present invention, it is possible to perform hybridization at extremely high speed in a normal gene expression analysis, and it becomes possible to perform hybridization at high speed without preparing a large amount of sample that had been conventionally required, if a low expressed gene is analyzed.

1. A micro-array substrate for a biopolymer, wherein a pair of two conduction paths connected to a direct-current or alternating-current source are installed on the substrate, in a part of a conduction path pattern is arranged the two conduction paths in proximity to each other to a degree such that an electric field distribution between the conduction paths becomes locally stronger, and probe molecules for biopolymer detection are immobilized on the conduction paths or close to its proximity part.

2. A micro-array substrate for a biopolymer, wherein a pair of two conduction paths connected to a direct-current or alternating-current source are installed on the substrate, in a part of a conduction path pattern is arranged the two conduction paths in proximity to each other to a degree such that an electric field distribution between the conduction paths becomes locally stronger, and probe molecules for biopolymer detection are immobilized on the conduction paths of its proximity part in an opposed substrate arranged opposite to said substrate, or close to its proximity part.

3. A micro-array substrate for a biopolymer according to claim 1, having two or more of said proximity parts.

4. A micro-array substrate for a biopolymer according to claim 1, wherein said substrate is a glass, a plastic, or a ceramic, and said two conduction paths are formed on the substrate by means of etching or printing.

5. A micro-array substrate for a biopolymer according to claim 1, wherein said conduction paths are insulated from a solution in areas other than an area immobilized with said probe molecules.

6. A micro-array substrate for a biopolymer according to claim 1, having an electrode for detecting the presence/absence of hybridization after hybridization, separately from said conduction paths.

7. A biopolymer hybridization device comprising a micro-array substrate for biopolymers according to claim 1, and a power source for applying either an AC voltage or DC voltage to two conduction paths set on said substrate, wherein
a voltage is applied from this power source to said conduction paths to generate an electric field, so that a sample biopolymer target contained in a solution on said substrate can be subjected to dielectrophoresis or electrophoresis, along this electric field.

8. A biopolymer hybridization device according to claim 7, wherein a cover substrate formed from a transparent material is provided opposite to a substrate surface set with said conduction paths, so that fluorescence from a hybridized biopolymer with fluorescent labeling can be observed through this cover substrate.

9. A biopolymer hybridization device according to claim 7, wherein said conduction paths are formed on a cover substrate formed from a transparent material, so that fluorescence from the hybridized biopolymer with fluorescent labeling can be observed from the back face of this cover substrate.

10. A method of performing hybridization of a biopolymer by using the device according to claim 7, comprising applying an alternating voltage or a direct voltage output from said power source between said conduction paths, to generate an electric field, so that a sample biopolymer target that is spontaneously dispersed in a solution is concentrated in the vicinity of the conduction paths by dielectrophoresis or electrophoresis.

11. A hybridization method for a biopolymer according to claim 10, comprising detecting said sample biopolymer target by means of fluorescent signals or electrical current value, after hybridization.
